(19) United States
(12) Patent Application Publication
(10) Pub. No.: US 2019/0076005 A1
(43) Pub. Date: Mar. 14, 2019

(54) FLUORESCENT IMAGING DEVICE FOR PLAQUE MONITORING AND MULTI-IMAGING SYSTEM USING SAME

(71) Applicant: INDUSTRIAL COOPERATION FOUNDATION CHONBUK NATIONAL UNIVERSITY, Jeollabuk-do (KR)

(72) Inventors: Chul Gyu SONG, Jeollabuk-do (KR); Sang Hun PARK, Chungcheongnam-do (KR)

(21) Appl. No.: 16/075,743

(22) PCT Filed: Feb. 1, 2017
(86) PCT No.: PCT/KR2017/001046

§ 371 Date: Nov. 16, 2018

(30) Foreign Application Priority Data
Feb. 5, 2016 (KR) 10-2016-0014697

(51) Int. Cl. A61B 1/04; A61B 5/00; A61B 5/02; G06T 5/50; A61B 8/08

(52) U.S. Cl. CPC A61B 1/043; A61B 5/0095; A61B 5/0071; G06T 2207/30101; G06T 5/50; A61B 8/0891; G06T 2207/20221; A61B 5/02007

(57) ABSTRACT

A fluorescent imaging device for plaque monitoring enables fluorescent imaging in a normal environment other than a dark room, thereby preventing inconvenience for both a medical staff and a patient due to the dark room. Also, a carotid artery plaque can be monitored in greater detail and in a short time by means of generating and combining a fluorescent image and a photo-acoustic image.

### TECHNICAL FIELD

The present invention relates to a technology for plaque monitoring, and more particularly, to a system and a method for monitoring plaque formed in a blood vessel.

### BACKGROUND ART

The number of patients dying of cardiovascular disease is the second highest in all cause of death from illness in Korea, whereas it is the highest in U.S. As such, there have been numerous researches on plaque monitoring for cardiovascular disease.

Plaque formed in a blood vessel causes vascular occlusion, resulting in severe conditions such as acute myocardial infarction. Especially, as plaque in carotid artery may cause stroke, cerebral infarction and so forth, plaque monitoring in carotid artery is very important.

A fluorescent imaging device is a device for collecting light generated by irradiating a fluorescent material with light. The fluorescent imaging device obtains an image of plaque by targeting fluorescent material to the plaque using a contrast agent.

Capturing a fluorescence image can only be done in the darkroom, which is inconvenient to both a medical staff and a patient. Therefore, there is a need for a method for improving such inconvenient.

### DISCLOSURE

### Technical Problem

The present invention was devised in order to solve the problem mentioned above. Purpose of the present invention is to provide an apparatus and method for capturing a fluorescence image even in a general environment other than a dark room through a filter and an image processing.

In addition, another purpose of the present invention is to provide a multi-imaging system and a method of combining a fluorescence image and a photo-acoustic image as a method for monitoring plaque in greater detail.

### Technical Solution

In order to achieve the purposes, an imaging system according to one embodiment of the present invention, comprises: a light source configured to irradiate infrared light to a blood vessel; a camera configured to generate a fluorescence image by receiving fluorescence emitted from fluorescent material targeted to plaque of the blood vessel; and a filter configured to filter light incident on the camera.

The filter may include LPF (Long Pass Filter) configured to block visible light and NF (Notch Filter) configured to block infrared light emitted from the light source.

The imaging system may further comprise an image processing unit configured to perform pseudo-coloring for the fluorescence image generated by the camera.

The imaging system may further comprise a laser configured to generate a laser pulse and irradiate the laser pulse to the blood vessel; a sensor configured to sense the photo-acoustic emitted from the blood vessel irradiated with the laser pulse; and a photo-acoustic generating unit configured to generate a photo-acoustic image using outcome sensing result of the sensor.

The imaging system may further comprise a combining unit configured to combine the fluorescence image and the photo-acoustic image.

The combining unit may further configured to combine the fluorescence image in horizontal plane, and the photo-acoustic image in vertical plane.

The laser pulse may be a laser pulse of a wavelength with the largest thermal expansion for main component of the plaque.

The wavelength of the laser pulse may be 1210 nm.

A method of plaque monitoring according to another embodiment of the invention comprises: irradiating infrared light to a blood vessel; filtering fluorescence from among the fluorescence and ambient light which are emitted from fluorescent material targeted to plaque in the blood vessel; and generating a fluorescence image using the filtered fluorescence.

### Advantageous Effects

According to the embodiments of the present invention, the present invention enables fluorescence imaging in a normal environment other than a dark room, thereby both a medical staff and a patient can be free of inconvenience caused in the dark room.

Also, according to the embodiments of the present invention, a carotid artery plaque can be monitored in greater detail and in a short time by means of generating and combining a fluorescence image and a photo-acoustic image.

### DESCRIPTION OF DRAWINGS

FIG. 1 is a view schematically illustrating an exterior of a fluorescent imaging device according to an embodiment of the invention.

FIG. 2 is a view illustrating a bottom plan view of the light source shown in FIG. 1.

FIG. 3 is a view illustrating a filter and a camera separated from the fluorescent imaging device shown in FIG. 1.

FIG. 4 is a flow chart illustrating pseudo-coloring process.

FIG. 5 is a flow chart illustrating calculation process of target efficiency.

FIG. 6 is a block diagram illustrating a multi-imaging system for plaque monitoring according to another embodiment of the invention.

FIG. 7 is a graph for describing laser absorption degree.

FIG. 8 is a view illustrating a structure of a multi-probe.

FIG. 9 is a view illustrating a light source and a laser irradiation structure.

FIG. 10 is a flow chart illustrating plaque monitoring method according to another embodiment of the present invention.

### MODES OF THE INVENTION

With the reference of the drawings, detailed description of the invention follows.

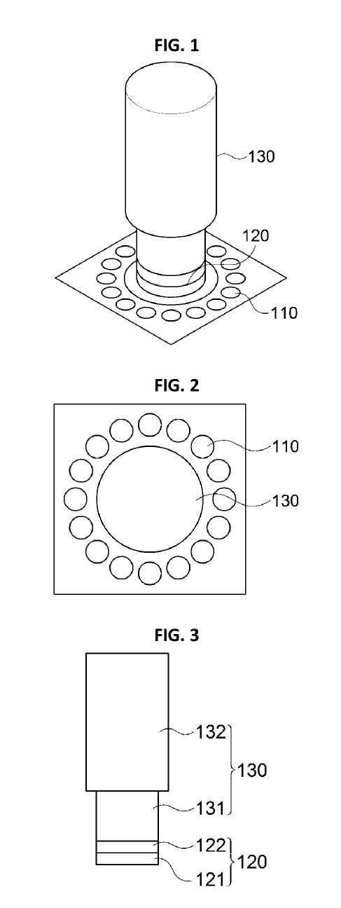

FIG. 1 is a view schematically illustrating an exterior of a fluorescence imaging device according to an embodiment of the invention. The fluorescence imaging device according to one embodiment of the present invention may capture fluorescence image of the carotid artery plaque even in a general environment other than a dark room.

The fluorescent imaging device includes a light source 110, a filter 120, and a camera 130.

The light source 110 continuously irradiates the carotid artery with near-infrared light. FIG. 2 is a view illustrating a bottom plan view of the light source 110 shown in FIG. 1. As shown in FIG. 2, a plurality of IR-LDs (Laser Diodes) are arranged on the circumference of the light source.

The near-infrared light irradiated from the light source 110 are absorbed into the fluorescent material targeted on the plaque using a contrast agent, whereby fluorescence is emitted from the excited fluorescent material.

In general, fluorescent material emits fluorescence of lower energy than that of absorbed light or electromagnetic waves.

The filter 120 is a means for limiting the light incident on the camera 130. In other words, the filter 120 allows only the fluorescence emitted from the fluorescent material to be incident on the camera.

In order to remove the light of the remaining wavelength other than the wavelength band of the fluorescence, the filter 120 is equipped with LPF (Long Pass Filter) 121 and NF (Notch Filter) 122 as shown in FIG. 3.

LPF 121 blocks visible light incident on the camera 130. Similarly, NF 122 blocks the near-infrared light irradiated from the light source 110 from being incident on the camera 130.

The camera 130 images the fluorescence passing through the filter 120 with an image pickup device such as a CCD sensor or a CMOS sensor. The CCD sensor or the CMOS sensor configured as an element sensitive to the wavelength of the fluorescence.

The camera 130 for fluorescent imaging comprises an optical system 131 and a camera body 132 as shown in FIG. 3. The fluorescence image generated by the camera 130 is displayed in real time through the monitor after being pseudo-colored, thereby a medical staff can monitor the plaque during surgery and emergency situations.

Fluorescent imaging can also be done in an operating room or an emergency room, not in a dark room, and as the light emitted from the light source 110 is near-infrared light that a patient or a medical staff cannot see, it does not disrupt surgery or emergency treatment.

Hereinafter, pseudo-coloring process for a fluorescence image generated by the camera 130 will be described in detail.

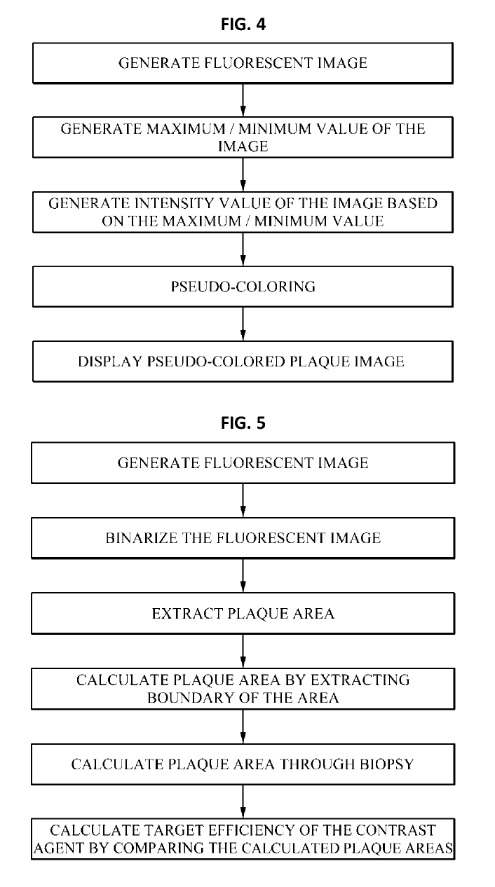

FIG. 4 is a flow chart illustrating pseudo-coloring process. Pseudo-coloring is an image processing that converts a black and white fluorescence image into arbitrary colors so that people can easily distinguish an object when they see the object. This can be performed by matching and converting intensity values of a gray scale image to Hue values of the HSV.

Specifically, the maximum/minimum value of a gray scale image to be pseudo-colored is obtained, and the intensity values of the image are normalized from 0 to 255 based on the maximum/minimum value. Then, H, S, and V images are combined into a HSV image from the normalized image and then the combined image is converted into RGB image.

Meanwhile, plaque target efficiency of the contrast agent can be calculated from the fluorescence image. FIG. 5 is a flowchart illustrating calculation process of the target efficiency.

As shown in FIG. 5, first, a fluorescence image is binarized and plaque area is extracted using a threshold. Next, only the boundary of the extracted area is extracted to calculate the area. Then, plaque area is calculated through biopsy of the blood vessel sample. Finally, the target efficiency of the contrast agent is calculated by comparing the two.

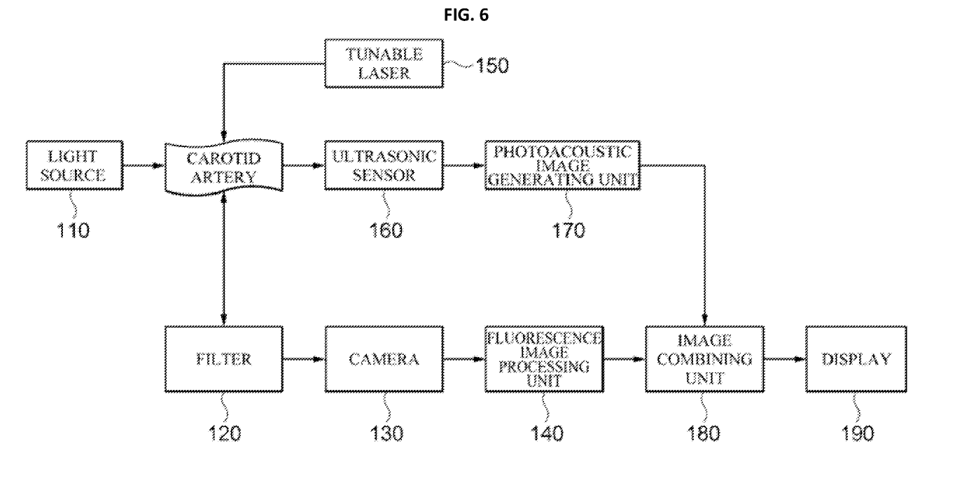

FIG. 6 is a block diagram illustrating a multi-imaging system for plaque monitoring in accordance with another embodiment of the present invention. The multi-imaging system according to the embodiment of the present invention is a system for monitoring plaque using a fluorescence image and a photo-acoustic image.

The fluorescence image is useful for planar observation/monitoring of plaque, and photo-acoustic imaging is useful for vertical monitoring/monitoring of plaque. Accordingly, using the multi-imaging system according to the embodiment of the present invention, the location and size of the plaque can be determined by the fluorescence image and the thickness of the plaque can be determined by the photo-acoustic image of the plaque. As a result, it is possible to accurately determine the shape of the plaque and the location of the plaque in the blood vessel.

As shown in FIG. 6, a multi-imaging system according to an embodiment of the present invention includes a light source 110, a filter 120, a camera 130, a fluorescence image processing unit 140, a tunable laser 150, an ultrasonic sensor 160, a photo-acoustic image generating unit 170, an image combining unit 180, and a display 190.

With regard to the light source 110, the filter 120 and the camera 130, as it has been described in detail in FIG. 1 to FIG. 3, a detailed description thereof will be omitted.

The fluorescence image processing unit 140 performs pseudo-coloring for the fluorescence image generated by the camera 130. The pseudo-coloring method is described above with regard to FIG. 4.

The tunable laser 150 may generate laser pulses of various wavelengths and irradiate the carotid artery with the laser pulses. The carotid artery is thermally expanded by absorbing the laser pulses irradiated by the tunable laser 150, and the photo-acoustic is emitted from the carotid artery by the thermal expansion.

Ultrasonic sensor 160 is a sensor for detecting the photo-acoustic emitted from the carotid artery. The result of photo-acoustic detection by the ultrasonic sensor 160 is applied to the photo-acoustic image generating unit 170.

The photo-acoustic image generating unit 170 generates a photo-acoustic image from the photo-acoustic detection result of the ultrasonic sensor 160. The image generated by the photo-acoustic image generating unit 170 is applied to the image combining unit 180.

The image combining unit 180 combines the fluorescence image pseudo-colored by the fluorescence image processing unit 140 and the photo-acoustic image generated by the photo-acoustic image generating unit 170. Specifically, the image combining unit 180 combines the fluorescence image and the photo-acoustic image by arranging the fluorescence image on the horizontal plane and arranging the photo-acoustic image on the vertical plane.

For this purpose, the optical axis of the camera 130 and the sensing axis of the ultrasonic sensor 160 need to be maintained as 90°.

The display 190 provides a medical staff with the plaque image combined by the image combining unit 180 by displaying the same.

Hereinafter, a principle and method for detecting carotid artery plaque using a photo-acoustic image are described in detail.

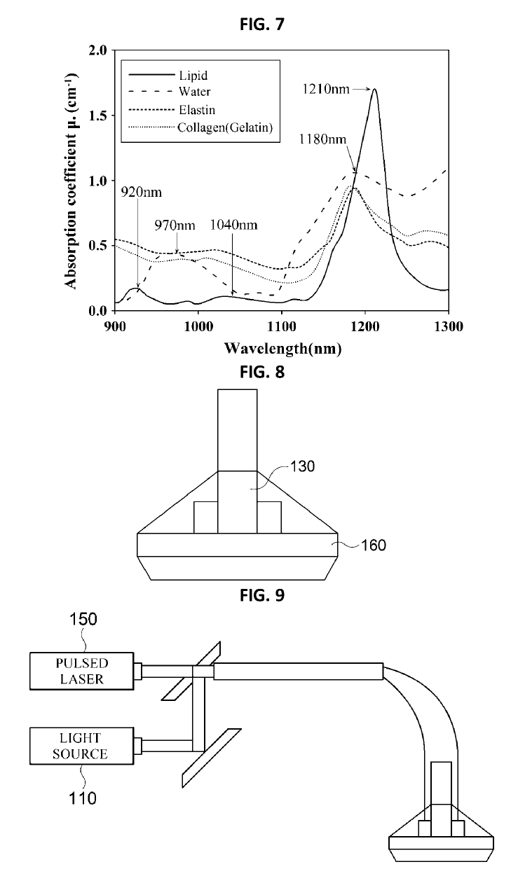

plaque is mainly composed of lipids. The photo-acoustic emitted from the lipids irradiated with the laser pulse of a specific wavelength is much greater than other tissues and thus the contrast is significant. In particular, as shown in FIG. 7, lipids have the highest absorbance for a laser of wavelength of 1210 nm, and emit the largest photo-acoustic.

FIG. 8 is a view illustrating a structure of a multi-probe including a light source 110, a filter 120, a camera 130, and an ultrasonic sensor 160. In FIG. 9, the near-infrared light emitted from the light source 110 and the pulse laser emitted from the tunable laser 150 are combined using a beam splitter and irradiated into the carotid artery through an optical fiber.

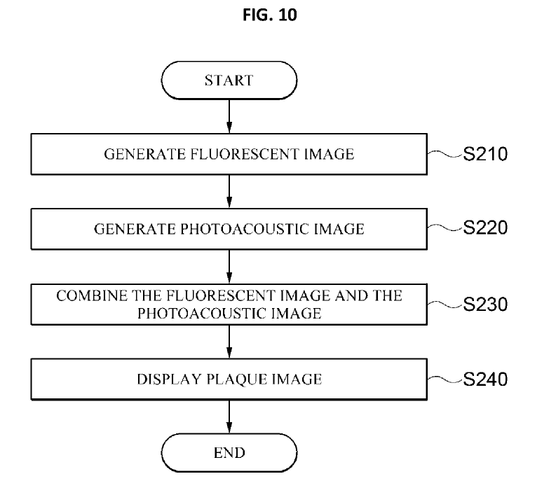

FIG. 10 is a flowchart illustrating plaque monitoring method according to another embodiment of the present invention.

As shown in FIG. 10, a fluorescence image is generated by irradiating near-infrared light to the fluorescent material targeted to the plaque (S210), and a laser pulse is irradiated to the plaque to generate a photo-acoustic image (S220).

Next, an image of plaque is generated (S230) by combining the fluorescence image and the photo-acoustic image generated in steps S210 and S220, respectively, and the generated image of plaque is displayed to a medical staff (S240).

As seen above, preferred embodiments of the plaque monitoring system and method have been described in detail.

In the embodiment described above, it is assumed that the light source 110 is provided in a fluorescent imaging device or in a multi-imaging system, but it is merely an example. The spirit of the present invention can also be applied to a case wherein the light source 110 is separated from the fluorescent imaging device or the multi-imaging system and implemented as a separate portable light source.

The plaque monitoring of carotid artery in the example above is merely illustrative. The spirit of the present invention can be applied to the plaque monitoring in other blood vessels than carotid artery.

While the present invention has been illustrated and described preferred embodiments above, the invention is not limited to the specific embodiments disclosed herewith; on the contrary, various modifications of embodiments can be made by those skilled in the art without departing from scope of the claims in the present invention. Moreover, these modifications should not be understood separate from the technical idea or perspective of the present invention.

1. An imaging system, comprising:
a light source configured to irradiate infrared light to a blood vessel;
a camera configured to generate a fluorescence image by receiving fluorescence emitted from fluorescent material targeted to plaque of the blood vessel; and
a filter configured to filter light incident on the camera

2. The imaging system of claim 1, wherein the filter comprises:
a Long Pass Filter (LPF) configured to block visible light; and
a Notch Filter (NF) configured to block the infrared light emitted from the light source.

3. The imaging system of claim 1, further comprising an image processing unit configured to perform pseudo-coloring for the fluorescence image generated by the camera.

4. The imaging system of claim 1, further comprising:
a laser configured to generate a laser pulse and irradiate the laser pulse to the blood vessel;
a sensor configured to sense photo-acoustic emitted from the blood vessel irradiated with the laser pulse; and
a photo-acoustic generating unit configured to generate a photo-acoustic image using a sensing result of the sensor.

5. The imaging system of claim 4, further comprising a combining unit configured to combine the fluorescence image and the photo-acoustic image.

6. The imaging system of claim 5, wherein the combining unit further configured to combine the fluorescence image in horizontal plane, and the photo-acoustic image in vertical plane.

7. The imaging system of claim 4, wherein the laser pulse is a laser pulse of a wavelength with the largest thermal expansion for main component of the plaque.

8. The imaging system of claim 7, wherein the wavelength of the laser pulse is 1210 nm.

9. A method of plaque monitoring comprising:
irradiating infrared light to a blood vessel;
filtering fluorescence from among the fluorescence and ambient light which are emitted from fluorescent material targeted to plaque in the blood vessel; and
generating a fluorescence image using the filtered fluorescence.
